(19) United States
(12) Patent Application Publication
(10) Pub. No.: US 2009/0312409 A1
(43) Pub. Date: Dec. 17, 2009

(54) LIPOPEXIA INHIBITOR AND FOOD OR BEVERAGE

(75) Inventors: Masaaki Yasue, Ibaraki (JP); Mari Yamamoto, Shizuoka (JP)

Correspondence Address: KNOBBE MARTENS OLSON & BEAR LLP, 2040 MAIN STREET, FOURTEENTH FLOOR, IRVINE, CA, 92614, US

(73) Assignees: Asahi Breweries, Ltd., Tokyo (JP); Incorporated Administrative Agency, National Agriculture and Food Research Organization, Ibaraki (JP)

(21) Appl. No.: 12/549,102

(22) Filed: Aug. 27, 2009

Related U.S. Application Data
(62) Division of application No. 11/911,711, filed on Jan. 22, 2008.

(30) Foreign Application Priority Data
Apr. 18, 2005 (JP) 2005-119858

(51) Int. Cl. A61K 31/352; A61P 3/06

(52) U.S. Cl. 514/456

(57) ABSTRACT

A lipopexia inhibitor which inhibits fat accumulation, comprising an effective amount of any methylated catechin having the following chemical formula (1) obtained by extraction of green tea or Pouchung tea, in which each of R₁, R₂, R₃ and R₄ independently is a hydrogen atom or methyl; and each of X₁ and X₂ independently is a hydrogen atom or hydroxyl.

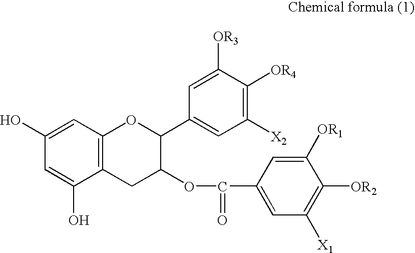

### CROSS-REFERENCE TO RELATED APPLICATIONS

This application is a divisional of application Ser. No. 11/911,711, filed Jan. 22, 2008, which is the U.S. National Phase filing under 35 U.S.C. §371 of PCT/JP2006/307858, filed Apr. 13, 2006, which designated the United States and was published in a language other than English, which claims priority under 35 U.S.C. § 119(a)-(d) to Japanese Patent Application No. 2005-119858, filed Apr. 18, 2005. The content of these applications is incorporated herein by reference in their entireties.

### BACKGROUND OF THE INVENTION

1. Field of the Invention

The present invention relates to antiobesity agents containing O-methylated catechin as an active ingredient or foods/beverages containing antiobesity agent.

2. Description of the Related Art

In recent years, the number of obese people has risen due to an increase in the amount of fat and calories in the diet. Upper-body obesity, abnormal glucose tolerance, high-neutral fat, and high-blood pressure are referred to as “deadly quartet”, and a combination of these is believed to accelerate the incidence of arteriosclerosis or cardiac disease, thereby threatening one's life. Since environmental factors such as dietary habit as well as genetic factors are greatly involved in the onset of these physiological abnormalities, prevention and amelioration thereof may often require a substantial change of lifestyle and may necessitate unsustainable self-restraint. Therefore, convenient means for prevention or amelioration of obesity that can be readily accepted by everyone has been anticipated.

To solve this problem, various useful substances have been found in foods or natural products, and research on functional foods utilizing their physiological functions have been extensively carried out. Functional foods are expected to improve blood lipid profile, blood-glucose level, blood pressure and the like, and to provide simple means for health-promotion and disease prevention, through being ingested daily by healthy humans. Among these, polyphenols have been drawing attention as a material that is expected to exhibit an effect to prevent or ameliorate obesity, and particularly green tea-derived catechins are currently regarded as one of the most promising materials under investigation.

Catechins are a type of tannin or polyphenol that are characteristic of green tea, and are one of the principal components which determines the taste of green tea. Catechins have been demonstrated to exhibit various effects such as antioxidant effects, arteriosclerosis suppressing effect, blood-pressure elevation suppressing effect, blood-glucose elevation suppressing effect etc. Accordingly, processes for preventing obesity have been known that utilize powder or the like of tea leaves as a raw material of healthy foods so as to raise the basal metabolism and to promote fat-burning, thereby preventing obesity (see, Non-patent Document 1).
Non-Patent Document 1: “Cha no Kagaku”, edited by Kei-ichiro Muramatsu, published by Asakura Publishing Co., Ltd., 1991

### SUMMARY OF THE INVENTION

### Problems to be Solved by the Invention

Although effectiveness of the catechins to promote combustion of fat and prevent obesity as described above has been conventionally known, a fat accumulation suppressor specifically containing methylated catechin has not yet been proposed.

The present invention was made in view of the foregoing problems, and an object of the invention is to provide a fat accumulation suppressor containing methylated catechin as an active ingredient, and a food/beverage containing the fat accumulation suppressor.

### Means for Solving the Problems

The present inventors conducted an extensive studied to achieve the object described above, and found that an O-methylated catechin, included in certain tea leaves, has an effect of suppressing fat accumulation. Accordingly, the invention as described below was accomplished.

More specifically, the present invention provides the following.

In a first aspect of the present invention, there is provided a fat accumulation suppressor including a constitutive effective amount of an O-methylated catechin that is obtained by extraction from at least one of green tea and paochong tea; and is represented by the following chemical formula (1):

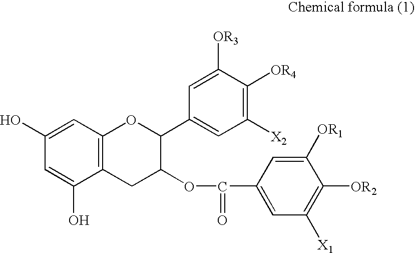

wherein R₁, R₂, R₃ and R₄ are each independently selected from the group consisting of a hydrogen atom and a methyl group, and X₁ and X₂ are each independently selected from the group consisting of a hydrogen atom and a hydroxy group.

According to the first aspect of the present invention, suppression of fat accumulation is enabled by allowing a beverage to include a constitutive effective amount of the O-methylated catechin. As described above, the catechins exhibit various effects such as the effect of an antioxidant, the effect of suppressing arteriosclerosis, the effect of suppressing blood-pressure elevation, the effect of suppressing elevation of blood-glucose, sterilization effects, antibacterial effects, deodorization effects etc.

The O-methylated catechins represented by the chemical formula (1) among others may be excellent in terms of antiallergic effect and neutral fat reduction. The reduction of neutral fat may inhibit the biosynthesis of remnant-like lipoprotein cholesterol and small-particle LDL-cholesterol, which leads to the prevention of arteriosclerosis, and also it may suppress neutral fat accumulation in adipocytes, which lowers the risk to develop obesity.

In addition, elevation of the neutral fat value in serum increases the risk of fatty liver and further the risk of hepatitis or liver cirrhosis, since neutral fat is incorporated into liver cells, following decomposition into fatty acid and glycerin by hepatic lipase; however, the neutral fat value can be lowered in accordance with the first aspect of the present invention by virtue of the constitutive effective amount of the O-methylated catechin included in the beverage, which may also make it possible to prevent the gallbladder/liver dysfunctions.

The term “fat accumulation suppressor” referred to herein means an agent that exhibits an effect of suppressing fat accumulation as described above. Moreover, the term “constitutive effective amount” refers to an amount in which the active ingredient that exhibits the effect of suppressing fat accumulation is estimated to achieve enough efficacy. Specifically, the O-methylated catechin may be included in an amount of 2 mg to 100 mg per 100 ml of beverage.

In a second aspect of the present invention, there is provided a fat accumulation suppressor according to the first aspect wherein the aforementioned O-methylated catechin is derived from leaves of tea selected from the varieties consisting of “Benifuuki”, “Benifuji”, “Benihomare”, “Yaeho”, “Surugawase”, “Yutakamidori”, “Kanayamidori”, “Okumusashi”, “Seishin-taipan”, “Seishin-oolong”, “Oba-oolong”, “Benibana”, “Benihikari”, “Yamakai”, “Yamamidori”, “Karabeni”, “Koushun”, “Souhuu” and “Okumidori”, and any mixture thereof.

According to the second aspect of the present invention, the O-methylated catechin is inherently found in the types of tea leaves of varieties such as “Benifuuki”, “Benifuji”, “Benihomare”, “Yaeho”, “Surugawase”, “Yutakamidori”, “Kanayamidori”, “Okumusashi”, “Seishin-taipan”, “Seishin-oolong”, “Benibana”, “Benihikari”, “Yamakai”, “Yamamidori”, “Karabeni”, “Koushun”, “Souhuu”, “Okumidori” and the like, and thus suppression of fat accumulation is enabled by the use of these tea leaves. These varieties of tea leaves are illustrative examples of tea leaves containing the O-methylated catechin, but not particularly limited thereto.

In a third aspect of the present invention, there is provided a fat accumulation suppressor according to either the first or second aspects of the present invention, in which the fat accumulation suppressor is labeled to indicate at least one of use for effective suppression of fat accumulation and that a constitutive effective amount of a fat accumulation suppressor is included.

According to the third aspect of the present invention, the labeling to indicate that the fat accumulation suppressor can be used for the effective suppression of fat accumulation and/or that a constitutive effective amount of a fat accumulation suppressor is included can allow consumers to have an impression of the efficacy as a fat accumulation suppressor according to the present invention.

In a fourth aspect of the present invention, there is provided a beverage containing 2 mg to 100 mg of the fat accumulation suppressor according to any one of the first to the third aspects of the present invention per 100 ml of the beverage.

According to the fourth aspect of the present invention, specifying the O-methylated catechin amount per 100 ml of the beverage in the range described above may make it possible to provide palatable beverages with a less bitter taste. When the amount of the included O-methylated catechin is above 100 mg, a higher “astringency” results leading to inappropriateness for beverages. On the other hand, when the amount is less than 2 mg, a sufficient effect cannot be achieved.

In a fifth aspect of the present invention, there is provided a food containing 100 mg to 5000 mg of the fat accumulation suppressor according to any one of the first to the third aspects of the present invention per 100 g of the food.

In the food, the amount of the included O-methylated catechin is preferably larger than that included in the beverage. According to the fifth aspect of the present invention, specifying the O-methylated catechin amount per 100 g of the food or a medicament in the range described above may make it possible to provide palatable food or medicament with a less bitter taste. Furthermore, when the amount of the included O-methylated catechin is above 5000 mg, a higher “astringency” results leading to inappropriateness for oral intake. On the other hand, when the amount is less of than 2 mg, sufficient antiobesity effect cannot be achieved.

In a sixth aspect of the present invention, there is provided a medicament containing 2 mg to 200 mg of the fat accumulation suppressor according to any one of the first to the third aspects of the present invention.

In the medicament, the amount of the included O-methylated catechin is preferably larger than that included in the food. According to the sixth aspect of the present invention, specifying the O-methylated catechin amount per tablet in the range described above may make it possible to provide a readily taken medicament with a less bitter taste.

In a seventh aspect of the present invention, there is provided a method of newly imparting a function of suppressing fat accumulation to a beverage by allowing the beverage to include a constitutive effective amount of the fat accumulation suppressor according to any one the first to the third aspects of the invention.

In an eighth aspect of the present invention, there is provided a functional beverage including a constitutive effective amount of an O-methylated catechin that is obtained by an extraction of at least one of green tea and paochong tea, and is represented by the following chemical formula (1):

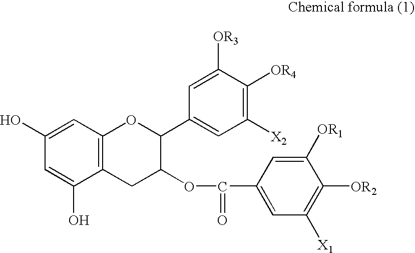

wherein R₁, R₂, R₃ and R₄ are each independently selected from the group consisting of a hydrogen atom and a methyl group, and X₁ and X₂ are each independently selected from the group consisting of a hydrogen atom and a hydroxy group,

the functional beverage being labeled to indicate at least one of: use for the effective suppression of fat accumulation; and that fat accumulation is suppressed.

### EFFECTS OF THE INVENTION

According to the functional beverage of the present invention, a food/beverage and a fat accumulation suppressor can be provided which suppress fat accumulation without inducing side-effects such as drowsiness, and have an universally accepted taste. Accordingly, prevention of lifestyle-related diseases such as hyperlipidemia, obesity, and liver disease is enabled by the daily action of drinking tea.

### DETAILED DESCRIPTION OF THE PREFERRED EMBODIMENT

The present invention will be explained in detail below.

### Production of Fat Accumulation Suppressor

The fat accumulation suppressor according to the present invention contains a constitutive effective amount of an O-methylated catechin component derived from certain tea leaves. The “O-methylated catechin” herein is represented by the chemical formula (1) and is a catechin which is methylated, and is an essential component in purification. It is preferred that the O-methylated catechin herein predominantly include epigallocatechin-3-O-(3-O-methyl)gallate (hereinafter, referred to as EGCG3″Me), epicatechin-3-O-(3-O-methyl)gallate (hereinafter, referred to as ECG3″Me), epicatechin-3-O-(4-O-methyl)gallate (hereinafter, referred to as ECG4″Me), epigallocatehin-3-O-(4-O-methyl)gallate (hereinafter, referred to as EGCG4″Me), gallocatechin-3-0-(3-O-methyl)gallate (hereinafter, referred to as GCG3″Me), catechin-3-O-(3-O-methyl)gallate (hereinafter, referred to as CG3″Me), and catechin-3-O-(4-O-methyl)gallate (hereinafter, referred to as CG4″Me), or gallocatechin-3-O-(4-O-methyl)gallate (hereinafter, referred to as GCG4″Me) and isomers thereof.

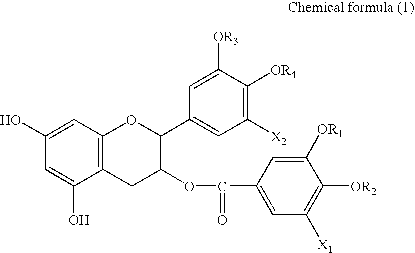

The fat accumulation suppressor according to the present invention is obtained by the extraction of the O-methylated catechin from the certain tea leaves in a conventionally known method. Examples of the certain tea leaves containing the O-methylated catechin include “Benifuuki”, “Benifuji”, “Benihomare”, “Yaeho”, “Surugawase”, “Yutakamidori”, “Kanayamidori”, “Okumusashi”, “Seishin-taipan”, “Seishin-oolong”, “Oba-oolong”, “Houoh-tanso”, “Houoh-suisen”, “Hakuyo-tanso-suisen”, “Oeko”, “Bui-suisen”, “Benibana”, “Benihikari”, “Yamakai”, “Yamamidori”, “Karabeni”, “Koshun” and “Okumidori”, and mixture thereof. These tea leaves may be used alone or as a mixture of two or more.

In addition, the extraction temperature is not particularly limited as long as it is higher than the melting point and lower than the boiling point of the solvent, which is preferably 10 to 100° C. in the case of water and 10 to 40° C. in the case of ethanol and methanol. The extracting time period is preferably from 10 seconds to 24 hours.

For example, dried tea leaves are processed into a powder by a pulverizing or milling process etc., then an extraction solvent is added to the powder, and the extract or its processed product is utilized. Examples of the extraction solvent include water; lower alcohols such as methanol, ethanol, propanol, isopropanol, butanol and isobutanol; ethers such as ethyl ether and dioxane; ketones such as acetone, and the like. Water, ethanol or a water-ethanol mixed solvent is preferred.

Although the resulting extract can be used neat as the fat accumulation suppressor according to the present invention, it is preferable to employ a method conventionally used as a chemical method for separation and purification. For example, liquid-liquid partition, thin-layer chromatography, absorption column chromatography, partition column chromatography, gel filtration chromatography, ion-exchange column chromatography, electrophoresis, high-performance liquid chromatography or the like can be employed. In addition, these methods for separation and purification may be employed in combination as needed.

The fat accumulation suppressor according to the present invention can be utilized for various applications such as beverages, medicament, foods and the like. As for the foods, it may be blended as a food additive into specified health foods, special nutritious foods, dietary supplements, health foods and the like. The subject food into which the additive is incorporated may be any of a variety of foods. As for beverages, it may be blended in beverages as specified health foods, special nutritious foods or dietary supplements; or into other nutritive beverages, healthy beverages, various kind of healthy tea and other beverages. As the other food products, confectioneries, breads, noodles, soybeans processed products, dairy products, egg processed products, fish cakes, fats and oils, seasonings and the like can be exemplified.

In a specific method of the production, an extract of the aforementioned certain tea leaves is used. In this method, pulverized tea leaves may be also blended. Moreover, a biochemically synthesized O-methylated catechin may be also blended.

Furthermore, for achieving a satisfactory effect of suppressing fat accumulation in beverages and foods by the O-methylated catechin as described above, additives such as antioxidants, flavors, various esters, organic acids, organic acid salts, inorganic acids, inorganic acid salts, inorganic salts, coloring agents, emulsifiers, preservatives, seasonings, sweetening agents, acidulants, fruit juice extracts, vegetable extracts, nectar extracts, pH regulators, quality stabilizers and the like can be blended alone or in combination.

Examples of the sweetening agent include sugar, glucose, fructose, corn syrup, glycyrrhizin, stevia, aspartame, fructo-oligosaccharide, galacto-oligosaccharide and the like. Examples of the acidulant include citric acid, tartaric acid, malic acid, lactic acid, fumaric acid, phosphoric acid and the like as well as fruit juices extracted from natural ingredients. It is favorable that citric acid or malic acid is included in a beverage in an amount of 0.1 to 5 g/L, preferably 0.5 to 2 g/L. Examples of the antioxidant include L-ascorbic acid, sodium L-ascorbate, erythorbic acid and sodium erythorbate. It is favorable that they are included in the beverage in an amount of 0.005 to 0.5% by mass, preferably 0.01 to 0.1% by mass.

The container used for the beverage can be provided, similarly to common beverages, in usual forms such as molded-containers based on polyethylene terephthalate (PET bottle, generally referred to), metal cans, paper containers combined with a metal foil or a plastic film, bottles and the like.

When the containers can be subjected to sterilization by heating after filling the containers as in the case of metal cans, the manufacture may be perfected under conditions defined by Food Hygiene Law. As for PET bottles or paper containers, which cannot be retort-sterilized, processes may be employed wherein the sterilization is conducted beforehand under similar sterilizing conditions as described above, for example, at a high temperature for a short period by use of a plate-type heat exchanger, followed by cooling to a certain temperature, and filling in the container. Further, another component may be aseptically blended to fill in a previously filled container. In addition, sterilization by heating under acidic conditions may be followed by aseptically adjusting the pH to be neutral again. Also, sterilization by heating under neutral conditions may be followed by aseptically adjusting the pH to be acidic again.

In addition, as the medicament including the fat accumulation suppressor according to the present invention as an active ingredient, those used for the treatment of allergic rhinitis, hyperlipidemia, obesity and liver disease may be exemplified.

With respect to the medicament, the composition according to the present invention can be orally administered neat, or after diluting with water or the like. Alternatively, it may be prepared through formulation with a known pharmaceutical carrier. For example, it can be administered as an orally administered liquid formulation such as a syrup or the like, or as an orally administered solid formulation such as tablets, capsules, granules, powders and the like, after processing it to give an extract, powder or the like followed by blending it with a pharmaceutically acceptable carrier. As the pharmaceutically acceptable carrier, a variety of organic or inorganic carrier materials can be employed which have been conventionally used as the material for formulation. Such a carrier material is blended as a diluting agent, a lubricant, a binder, a disintegrant or the like in solid formulations, while as a solvent, a diluting agent, a suspending agent, a binder or the like in liquid formulations. Moreover, additives for formulations such as an antiseptic agent, an antioxidant, a coloring agent, a sweetening agent and the like can be used as needed.

### EXAMPLES

### Example 1

### Production of Tablet

“Benifuuki” was subjected to extraction at 90° C. using purified water in an amount 30 times of the tea leaves to produce an extracted liquid. The O-methylated catechin component in the extract was purified by absorption and desorption to a synthetic resin with an organic solvent. This purified product in an amount of 1 g, 19 g of powdered “Benifuuki”, 60 g of lactose and 15 g of magnesium stearate were mixed homogeneously, and formed into a tablet and a capsule by an ordinary method.

### Example 2

### Production of Capsule

200 mg of the purified product obtained by the method according to Example 1 was encapsulated to give a capsule.

### Example 3

### Production of Powder

2 g of the purified product obtained by the method according to Example 1, 78 g of powdered tea leaves of “Benifuuki”, each 10 g of starch and lactose were mixed homogeneously, and formed into a powder by an ordinary method.

### Example 4

### Production of Candy

2 g of the purified product obtained by the method according to Example 1, 8 g of powdered tea leaves of “Benifuuki”, 20 g of sucrose, 60 g of starch syrup (solid content: 75%), 9.5 g of water, 0.45 g of a coloring agent and 0.05 g of a flavor were formed into a candy by an ordinary method.

### Example 5

### Production of Cookie

2 g of the purified product obtained by the method according to Example 1, 8 g of powdered tea leaves of “Benifuuki”, 32 g of soft flour, 16 g of whole egg, 16 g of butter, 25 g of sugar, 10.8 g of water, and 0.2 g of baking powder were formed into a cookie by an ordinary method.

### Example 6

### Production of Beverage

“Benifuuki” tea leaves were subjected to extraction at 90° C. using purified water in an amount 30 times of the tea leaves to produce an extracted liquid. To the resulting extract a water regulator was added, such as sodium bicarbonate and vitamin C, and mixed therewith. The mixture was sterilized, and filled in a hermetic container with full nitrogen, to yield a sample beverage 1.

### Example 7

### Study of Effect of Suppressing Accumulation of Neutral Fat in Cultured Adipocyte

The O-methylated catechin component was purified by the method of Sano (J. Agrlc. Food. Chem. 1999, 47, 1906-1910). More specifically, in this method, extraction with an organic solvent and purification with an HP-20 column are conducted. Adipose tissue around the testis of a male SD rat weighing 250 g was collected, and the tissue was treated with collagenase and hyaluronidase to disperse the cells by an ordinary method, whereby a cell suspension was prepared. A group of mature adipocytes floating after centrifugal separation, and a group of adipocyte precursor cells precipitated after the centrifugal separation were separately collected. The former was cultured in a flask, while the latter was cultured in a 96-well culture plate (30000 cells/0.1 ml medium/well), each for 3 days.

Subsequently, the culture supernatant of the mature adipocyte was collected to which the O-methylated catechin component was added to give a certain concentration. Thereafter, each 0.1 ml of the mixture was added to wells of the precursor cells. The precursor cells were further cultured for additional 4 days, and then Alamar Blue solution was added to half of these wells followed by culturing for 2 hours. Then, viability of the cells was determined. As for the remaining wells, the concentration of neutral fat in the cells was determined after washing. The results are shown in Table 1.

TABLE 1

| Total amount of O- | Accumulation of neutral fat | |
|---|---|---|
| methylated catechins (μg/ml) | (%) | Viability (%) |
| 0.0 (non-addition) | 100 | 100 |
| 3.125 | 97 | 112 |
| 6.25 | 94 | 106 |
| 12.5 | 86 | 105 |
| 25.0 | 76 | 88 |

Accordingly, it was demonstrated that the O-methylated catechin component suppressed the accumulation of neutral fat in a concentration dependent manner without affecting the viability of the adipocyte precursor cells.

### Example 8

### Anti-Obesity Effect of “Benifuuki” Tea Leaves

Four-week old male ICR mice were divided into three groups consisting of 8 animals respectively, and each group was freely fed with a purified chow (AIN-93G), a purified chow including 1% powdered “Yabukita” tea leaves, and a purified chow including 0.5% “Benifuuki” tea leaves, respectively, for 7 weeks. After completing this period, the test groups were compared on the body weight, the weight of the testicular fat, and the concentration of neutral fat in the liver. The amount of “Benifuuki” tea leaves to the chow was half of the amount of “Yabukita” tea leaves for adjusting the total amount of catechins. The results are shown in Table 2.

TABLE 2

| | | Fat around | Concentration of |
|---|---|---|---|
| | Body weight | the testis | neutral fat in the liver |
| Test groups | (g) | (g) | (mg/g) |
| Usual purified chow | 52.3 ± 2.0 | 2.1 ± 0.3 | 163 ± 20 |
| 1% “Yabukita” tea | 49.7 ± 3.6 | 1.8 ± 0.3 | 138 ± 16 |
| leaves | | | |
| 0.5% “Benihuuki” | 47.2 ± 2.1 | 1.6 ± 0.2 | 114 ± 17(*) |
| tea leaves | | | |

(*)p < 0.05 vs. Usual chow groups

As shown in Table 2, “Benifuuki” tea leaves exhibited significantly greater effects of lowering the body weight, the weight of the fat around the testis, and the concentration of neutral fat in the liver as compared with “Yabukita” tea leaves not containing the O-methylated catechin.

What is claimed is:

1. A method of suppressing fat accumulation in an individual, comprising:
identifying an individual in need of suppression of fat accumulation; and
administering an effective fat-suppressing amount of a methylated catechin according to Chemical Formula (1) below to said individual 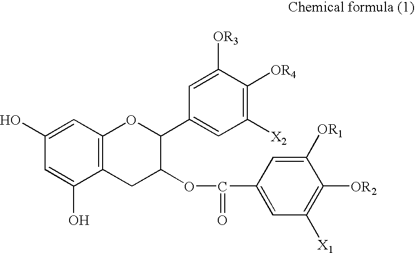
wherein R₁, R₂, R₃ and R₄ are each independently selected from the group consisting of a hydrogen atom and a methyl group with the proviso that at least one of R₁, R₂, R₃ or R₄ is a methyl group, and X₁ and X₂ are each independently selected from the group consisting of a hydrogen atom and a hydroxyl group.

2. The method of claim 1, wherein said methylated catechin is derived from at least one tea leaf selected from the group consisting of Benifuuki, Benifuji, Benihomare, Yaeho, Surugawase, Yutakamidori, Kanayamidori, Okumusashi, Seishin-taipan, Seishin-oolong, Ohba-oolong, Benibana, Benihikari, Yamakai, Yamamidori, Karabeni, Koushun, Souhuu, Okumidori and mixtures thereof.

3. The method of claim 1, wherein said methylated catechin is selected from the group consisting of epigallocatechin-3-O-(3-O-methyl)gallate, epicatechin-3-O-(3-O-methyl) gallate, epigallocatechin-3-O-(4-O-methyl)gallate, gallocatechin-3-O-(3-O-methyl)gallate, catechin-3-O-(3-O-methyl)gallate, catechin-3-O-(4-O-methyl)gallate, gallocatechin-3-O-(4-O-methyl)gallate and mixtures thereof.

4. The method of claim 1, wherein said methylated catechin is administered to said individual in a medicament comprising from 2 mg to 5000 mg of said methylated catechin.

5. The method of claim 4, wherein said medicament comprises from 2 mg to 200 mg of said methylated catechin.

6. The method of claim 1, wherein said methylated catechin is administered in a food.

7. The method of claim 6, wherein said methylated catechin is administered in a food, wherein the food provides 100 mg to 5000 mg of the methylated catechin per 100 g of the food.

8. The method of claim 1, wherein said methylated catechin is administered in a beverage, wherein the beverage provides 2 mg to 100 mg of the methylated catechin per 100 ml of the beverage.

9. The method of claim 1, wherein said methylated catechin is a separated and purified methylated catechin.
